(19) United States
(12) Patent Application Publication
(10) Pub. No.: US 2020/0281685 A1
(43) Pub. Date: Sep. 10, 2020

(54) FUNCTIONAL IMAGING OF SURGICAL SITE WITH A TRACKED AUXILIARY CAMERA

(71) Applicant: Covidien LP, Mansfield, MA (US)

(72) Inventor: Dwight MEGLAN, Westwood, MA (US)

(21) Appl. No.: 16/645,075

(22) PCT Filed: Sep. 6, 2018
(86) PCT No.: PCT/US18/49655

§ 371 Date: Mar. 6, 2020

Related U.S. Application Data
(60) Provisional application No. 62/556,009, filed on Sep. 8, 2017.

(51) Int. Cl. A61B 90/00; A61B 1/04; A61B 1/313; H04N 5/265; H04N 5/225; H04N 5/232; G06T 7/70

(52) U.S. Cl. CPC A61B 90/361; G06T 2207/20212; A61B 1/313; H04N 5/265; H04N 5/2257; H04N 5/23229; G06T 7/70; A61B 2090/373; A61B 2090/364; A61B 2090/376; A61B 2090/378; H04N 2005/2255; G06T 2207/30244; G06T 2207/10068; A61B 1/04

(57) ABSTRACT

A surgical imaging system includes a camera, a first imager, and a processing unit. The camera is configured to capture optical images of a surgical site along a first optical path. The first imager is configured to capture first functional images of the surgical site along a second path that is separate from the first optical path. The processing unit is configured to generate a combined view of the surgical site from the captured first functional images and the captured optical images and to transmit the combined view to a display.

### BACKGROUND

During surgical procedures cameras can be used to visualize a surgical site. Particularly, in minimally invasive surgery (MIS), including robotic surgery, specialized optical cameras can be used to allow a surgeon to visualize a surgical site.

To understand the functional aspects of tissue at the surgical site, that are not readily observable, e.g. blood flow being present in subsurface tissue or that certain tissues are cancerous, these specialized cameras and specialized imaging protocols have been developed. When these specialized imaging techniques are used in conjunction with a typical white light-based endoscope, in minimally invasive or robotic surgery, a specially constructed endoscope is used that can allow both visible light, as well as functional imaging derived data, to be recorded from the same point of view. This type of endoscope typically is quite expensive.

Thus, there is a need to develop systems that allow for optical and functional imaging of surgical sites that can be used with existing white light-based endoscopes without requiring specially constructed endoscopes.

It would be desirable to develop and use a stand-alone camera that can be readily positioned inside the body, to provide this additional functional imaging information, and then overlay that additional functional imaging information upon the existing main endoscope image, allowing the capabilities of current endoscopes to be extended without need for a specially constructed endoscope.

### SUMMARY

In an aspect of the present disclosure, a surgical imaging system includes a camera, a first imager, and a processing unit. The camera is configured to capture optical images of a surgical site along a first optical path. The first imager is configured to capture first functional images of the surgical site along a second path that is separate from the first optical path. The processing unit is configured to generate a combined view of the surgical site from the captured first functional images and the captured optical images and to transmit the combined view to a display.

In aspects, the system includes a display that is configured to receive the combined view of the captured first functional and optical images and to display the combined view. The system may include an endoscope that is configured to pass through an opening to access a surgical site. The camera may be disposed within the endoscope. The first imager may be releasably coupled to an outer surface of the endoscope. The first imager may include a lead that extends along an outer surface of the endoscope to couple the first imager to the processing unit. The lead may be configured to supply power to the first imager and/or to transmit captured first functional images to the processing unit. The endoscope may include a switch that is movable between a first position in which only the optical images are transmitted to the display and a second position in which the combined view is transmitted to the display.

In some aspects, the system includes a second imager that is configured to capture second functional images of the surgical site along a third path that is separate from the first optical path and second path. The processing unit may be configured to receive and combine the captured second functional images with the captured optical images and to transmit the combined view to the display. The processing unit may be configured to combine the captured first and second functional images with the captured optical images and to transmit the combined view to the display.

In certain aspects, the processing unit is configured to determine the pose of the camera from the optical images captured by the camera and to determine the pose of the first imager from the first functional images captured by the first imager. The processing unit may be configured to generate a combined view based on the pose of the first imager relative to the pose of the camera.

In another aspect of the present disclosure, a method of displaying views of a surgical site on a display with a processing unit includes, receiving optical images of a surgical site along a first optical path from a camera, receiving first functional images of the surgical site along a second path that is separate from the first optical path form a first imager, combining the first functional images and the optical images of the surgical site into a combined view, and transmitting the combined view to a display.

In aspects, combining the first functional and optical images includes locating a common object in each of the first functional images and the optical images to position the first functional images over the optical images. Additionally or alternatively, the method may include receiving a pose of the camera and receiving a pose of the first imager and combining the first functional and optical images may include position the first functional images over the optical images based on the pose of the first imager relative to the pose of the camera.

In some aspects, the method includes receiving second functional images of the surgical site along a third path that is separate from the first optical path and second path from a second imager. Combining the first functional images and the optical images of the surgical site into the combined view may further include combining the second functional images with the first functional images and the optical images. The method may include extending a field of view of the camera with the second imager.

In another aspect of the present disclosure, a method of visualizing a surgical site on a display includes positioning a camera within a surgical site to capture optical images along a first optical path, positioning a first imager within the surgical site to capture first functional images along a second path separate from the first optical path, and viewing a combined view of the first functional images overlaid of the optical images on a display.

In aspects, the method includes position the first imager within the surgical site with a surgical instrument. Positioning the first imager may include position the first imager on an outer surface of an endoscope supporting the camera. The method may include actuation a switch to activate the combined view before viewing the combined view.

Further details and aspects of exemplary embodiments of the present disclosure are described in more detail below with reference to the appended figures.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a surgical imaging system in accordance with the present disclosure including an optical imaging system, a processing unit, a functional imaging system, and a display;

FIG. 2 is a cut-away of the detail area shown in FIG. 1 illustrating a camera of the optical imaging system shown in FIG. 1 and imagers of the functional imaging system shown in FIG. 1 within a body cavity of a patient; and

FIG. 3 is a flowchart of a method of displaying a combined view of functional images and optical images on a display with a processing unit in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician. In addition, as used herein the term “pose” is understood to mean a position and orientation of an object in space.

This disclosure generally relates to surgical systems including a camera capturing optical images of a surgical site and one or more stand-alone functional imagers which capture functional images of the surgical site. The functional images may be overlaid or painted over the optical images to be viewed simultaneously with the optical images on a display. The functional imagers may be disposed within the surgical site separate from the camera such that the functional imagers are disposed along separate imaging paths from the camera. The surgical system may include a processing unit that uses the pose of the functional imager relative to the camera to combine functional image data with optical images. The processing unit uses objects within the field of view of the camera and imagers, e.g., tissue structures or surgical instruments, to combine functional images with optical images.

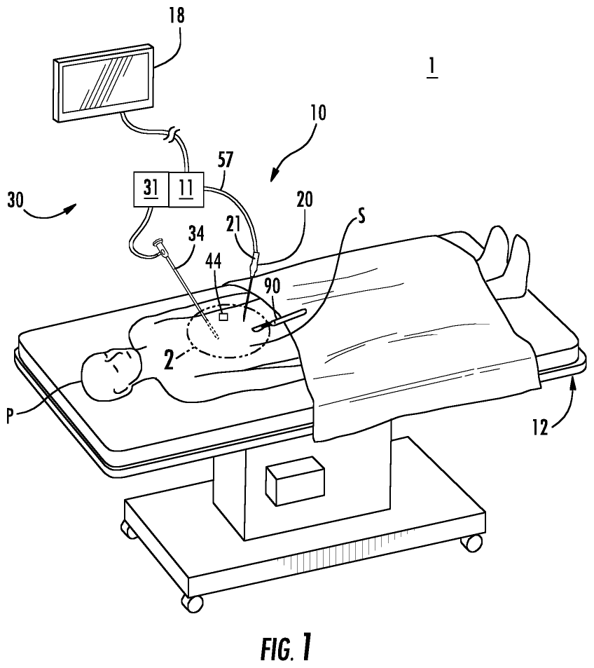

Referring now to FIG. 1, a surgical system 1 provided in accordance with the present disclosure includes an optical imaging system 10 and a functional imaging system 30. The optical imaging system 10 includes a processing unit 11, a display 18, and an endoscope 20. As shown, the surgical system 1 is a laparoscopic surgical system; however, the surgical system 1 may be an endoscopic surgical system, an open surgical system, or a robotic surgical system. For a detailed description of a suitable robotic surgical system, reference may be made to U.S. Pat. No. 8,828,023, the entire contents of which are incorporated herein by reference.

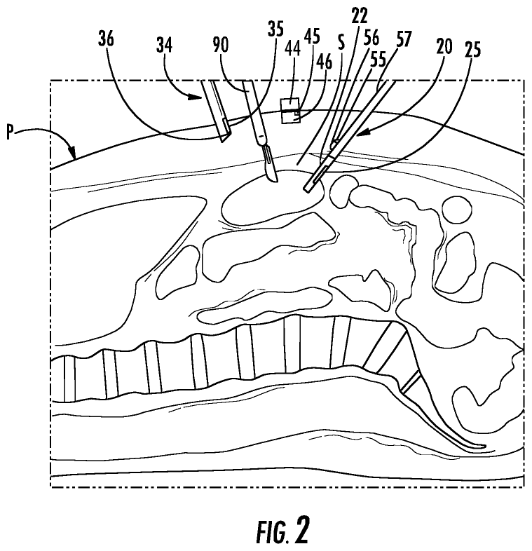

With additional reference to FIG. 2, the optical imaging system 10 is configured to provide optical views or images of a surgical site “S” within a body cavity of a patient “P” and to transmit the optical images to the display 18. The endoscope 20 of the optical imaging system 10 includes a camera 22 to capture optical images of the surgical site “S” during a surgical procedure as detailed below.

The endoscope 20 is inserted through an opening, either a natural opening or an incision, to position the camera 22 within the body cavity adjacent the surgical site “S” to allow the camera 22 to capture optical images of the surgical site “S”. The camera 22 transmits the captured optical images to the processing unit 22. The processing unit 11 receives optical images or data of the surgical site “S” from the camera 22 and displays the optical images on the display 18 such that a clinician can visualize the surgical site “S”. The endoscope 20 and/or camera 22 includes a sensor 25 (FIG. 2) that captures the pose of the camera 22 as the optical images of the surgical site “S” are captured. The sensor 25 is in communication with the processing unit 11 such that the processing unit 11 receives the pose of the camera 22 from the sensor 25 and associates the pose of the camera 22 with the optical images captured by the camera 22.

With continued reference to FIGS. 1 and 2, the functional imaging system 30 includes a control unit 31 and one or more functional imagers, e.g., imager 36. The control unit 31 may be integrated with or separate from the processing unit 11. The functional imaging system 30 may include a probe 34 that is inserted through an opening to support the imager 36 within the body cavity of the patient “P” to position the imager 36 adjacent the surgical site “S”. The imager 36 may also be positioned within the surgical site “S” with a surgical instrument, e.g., surgical instrument 90. The imager 36 captures functional images of the surgical site “S” which may include, but are not limited to, optical images, IR images, X-Ray images, fluorescence images, photoacoustic images, multi/hyper-spectral, ultrasound, or Cerenikov radiation. The functional images may provide information that is not observable with the camera 22 of the endoscope 20, e.g., blood flow in subsurface tissue, cancerous tissue, or optical images outside a field of view of the camera 22. The imager 36 transmits the functional images to the control unit 31. The probe 34 and/or the imager 36 includes a sensor 35 that captures the pose of the imager 36 as the functional images of the surgical site “S” are captured and transmits the pose of the imager 36 to the control unit 31. Specifically, the sensor 35 may use objects within the field of view of the imager 36 to capture the pose of the imager 36.

The control unit 31 receives the functional images from the imager 36 and the pose of the imager 36 as the functional images are captured from the sensor 35 and generates functional image data from the images and pose. The control unit 31 transmits the functional image data to the processing unit 11 which receives the functional image data from the control unit 31 and combines the functional image data with the optical images from the camera 22. In some embodiments, the imager 36 and/or the sensor 35 are in direct communication with the processing unit 11 such that the control unit 31 may be unnecessary.

To combine the functional image data from the imager 36 with the optical images from the camera 22, the processing unit 11 analyzes the optical images and the functional images to align the functional images with the optical images. The processing unit 11 may locate a common structure within the surgical site “S” within the optical images and the functional images to align the functional images with the optical images. Specifically, the processing unit 11 may identify an optical path of the camera 22 from a position of the common structure within the optical images and identify an optical path of the imager 36 from the position of the common structure within the functional images. With the optical paths identified, the processing unit 11 transforms the optical path of the imager 36 to align with the optical path of the camera 22 to overlay the functional images with the optical images. For example, a surgical instrument may be captured in the functional images and in the optical images such that the surgical instrument may be used to identify and align the optical paths of the functional images with the optical images. Additionally or alternatively, a structure within the surgical site “S”, e.g., an organ, an implant, etc., may be used in a similar manner to a surgical instrument. It will be appreciated that the functional images and the optical images undergo a spatial operation to combine the two-dimensional images into a composite of three-dimensional information.

The pose of the camera 22 and the pose of the imager 36 may also be used to align the functional images with the optical images in combination with or separate from a common structure within the surgical site “S”. When the functional images are aligned with the optical images, the processing unit 11 may overlay or paint the optical images with the functional image data on the display 18 such that a clinician can view the functional image data simultaneously with the optical images on the display 18. The endoscope 20 may include a selector or switch 21 which allows a clinician to selectively view the functional image data with the optical images of the surgical site “S”.

Continuing to refer to FIG. 2, the functional imaging system 30 may include a functional imager 46 entirely or substantially entirely disposed within the body cavity of the patient “P” adjacent the surgical site “S”. Similar to the functional imager 36, the functional imager 46 may include a sensor 45 to capture the pose of the imager 46 as the functional imager 46 captures functional images of the surgical site “S”. The functional imager 46 and sensor 45 transmit functional images and the pose of the imager 46, respectively, to the control unit 31. The control unit 31 combines the functional images with the pose of the imager 46 to generate functional image data which is transmitted to the processing unit 11.

The imager 46 may be magnetically coupled to a base 44 disposed on a surface of the patient “P” outside of the body cavity. Manipulation of the base 44 along the surface of the patient “P” may move the imager 46 within the body cavity of the patient “P”. In addition, the imager 46 and/or the sensor 45 may transmit data to the base 44 such that the base 44 relays the data to the control unit 31.

The processing unit 11 may combine the functional image data from imager 46 with the functional image data from camera 36 and simultaneously overlay both sets of functional image data with the optical images from the camera 22. Additionally or alternatively, the processing unit 11 may allow a clinician to select which functional image data, if any, to overlay the optical images from the camera 22. The functional imaging system 30 may move the functional imager 46 around the surgical site “S” to record information at a plurality of locations within the surgical site “S” such that the functional image data can be “painted” over the optical images during a procedure.

Still referring to FIG. 2, the functional imaging system 30 may include an imager 56 releasably coupled to or disposed within a wall of the endoscope 20. The imager 56 is similar to the imager 36 detailed above and as such, the similarities between the imager 56 and imager 36 will not be described in detail for reasons of brevity. The imager 56 may extend out of the endoscope 20 or be placed on the endoscope 20 by a surgical instrument, e.g., surgical instrument 90, during a surgical procedure. The imager 56 may include a sensor 55 or the sensor 25 of the endoscope 20 may capture the pose of the imager 56 as functional images from the imager 56 are captured. The imager 56 may include leads 57 that extend along the endoscope 20 to electrically connect the imager 56 to the processing unit 11. The leads 57 may deliver power to the imager 56 and transmit data from the imager 56 to the processing unit 11 or the control unit 31.

As detailed above, the imagers, e.g., imagers 36, 46, 56, may capture optical images of the surgical site “S” including data outside of a field of view of the camera 22. The processing unit 11 may combine the optical images from the imagers 36, 46, 56 with the optical images from the camera 22 for viewing on the display 18 such that a clinician can visualize an extended field of view of the surgical site “S” beyond what is possible with the camera 22 alone. In addition, when multiple imagers are utilized, one of the imagers, e.g., imager 56, may provide optical images and the other imagers, e.g., imagers 36, 46, may provide functional images which can be overlaid with the optical images of both the camera 22 and the optical images from the other imagers, e.g., imager 56.

The imagers, e.g., imagers 36, 46, 56, may be in wireless communication with the processing unit 11 and/or the control unit 31. The wireless communication may be radio frequency, optical, WIFI, Bluetooth® (an open wireless protocol for exchanging data over short distances (using short length radio waves) from fixed and mobile devices, ZigBee® (a specification for a suite of high level communication protocols using small, low-power digital radios based on the IEEE 802.15.4-2003 standard for wireless personal area networks (WPANs)), Ultra wideband radio (UWB), etc.

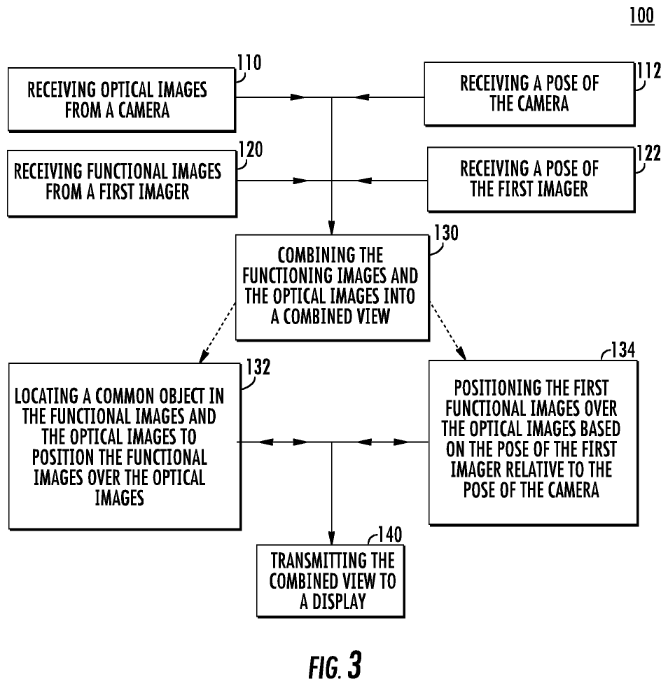

With reference to FIG. 3, a method 100 of displaying a combined view of functional images and optical images on a display with a processing unit is described in accordance with the present disclosure with reference to the surgical system 1 of FIGS. 1 and 2. Initially, the processing unit 11 receives optical images from the camera 22 (Step 110) and may receive a pose of the camera 22 when the optical images were captured (Step 112). The processing unit 1 also receives functional images from the imager 36 (Step 120) and may receive a pose of the imager 36 when the functional imagers were captured (Step 122). It will be appreciated that Steps 110, 112, 120, and 122 may occur in parallel or serially.

As the processing unit 11 receives the optical images and the functional images, the processing unit 11 combines the functional images with the optical images (Step 130). As detailed above, the processing unit 11 may identify or locate a common object in the optical images and the functional images to identify an optical path or pose of the imager 36 relative to the pose of the camera 22 (Step 132). The processing unit 11 may then transform the functional images to the optical path of the camera 22 such that the functional images overlay the optical images. Additionally or alternatively, the processing unit 11 may use the pose of the camera 22 and the pose of the imager 36 received in Steps 112, 122 above to transform the functional images to the optical path of the camera 22 (Step 134). In some embodiments, the processing unit 11 performs Step 134 and fine tunes the transformation by subsequently performing Step 132.

After combining the functional images and the optical images, the processing unit 11 transmits the combined view to the display 18 for visualization by a clinician. It will be appreciated that the combined view is transmitted in substantially real-time to increase the situational awareness of a clinician during a surgical procedure.

It will be appreciated that utilizing stand-alone functional cameras in combination with an endoscope increases the visualization of a surgical site with a reduced cost to utilizing a single specialized endoscope having optical and functional cameras integrated together. In addition, by allowing for multiple functional imagers to be disposed within a surgical site, a clinician may extend the field of view of the surgical site. Further, each functional imager may provide functional views of the surgical site which may be selectively overlaid with optical images provided by the endoscope providing a clinician with greater flexibility of visualization during a surgical procedure. By increasing visualization, extending the field of view of a surgical site, and providing greater flexibility of visualization surgical outcomes may be improved, surgical times may be reduced, and/or the cost of surgical procedures may be reduced.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A surgical imaging system comprising:
a camera configured to capture optical images of a surgical site along a first optical path;
a first imager configured to capture first functional images of the surgical site along a second path separate from the first optical path; and
a processing unit configured to generate a combined view of the surgical site from the captured first functional images and the captured optical images and to transmit the combined view to a display.

2. The system according to claim 1, further comprising a display configured to receive the combined view of the captured first functional and optical images and to display the combined view.

3. The system according to claim 1, further comprising an endoscope configured to pass through an opening to access a surgical site.

4. The system according to claim 3, wherein the camera is disposed within the endoscope.

5. The system according to claim 3, wherein the first imager is releasably coupled to an outer surface of the endoscope.

6. The system according to claim 3, wherein the first imager includes a lead extending along an outer surface of the endoscope to couple the first imager to the processing unit, the lead configured to at least one of supply power to the first imager or transmit captured first functional images to the processing unit.

7. The system according to claim 1, further comprising a second imager configured to capture second functional images of the surgical site along a third path separate from the first optical path and second path, the processing unit configured to receive and combine the captured second functional images with the captured optical images and to transmit the combined view to a display.

8. The system according to claim 7, wherein the processing unit is configured to combine the captured first and second functional images with the captured optical images and to transmit the combined view to a display.

9. The system according to claim 1, wherein the processing unit is configured to determine the pose of the camera from the optical images captured by the camera and to determine the pose of the first imager from the first functional images captured by the first imager, and wherein the processing unit is configured to generate the combined view based on the pose of the first imager relative to the pose of the camera.

10. A method of displaying views of a surgical site on a display with a processing unit, the method comprising:
receiving optical images of a surgical site along a first optical path from a camera;
receiving first functional images of the surgical site along a second path, separate from the first optical path, from a first imager;
combining the first functional images and the optical images of the surgical site into a combined view; and
transmitting the combined view to a display.

11. The method according to claim 10, wherein combining the first functional and optical images includes locating a common object in each of the first functional images and the optical images to position the first functional images over the optical images.

12. The method according to claim 10, further comprising receiving a pose of the camera and receiving a pose of the first imager, wherein combining the first functional and optical images includes positioning the first functional images over the optical images based on the pose of the first imager relative to the pose of the camera.

13. The method according to claim 10, further comprising receiving second functional images of the surgical site along a third path, separate from the first optical path and second path, from a second imager.

14. The method according to claim 13, wherein combining the first functional images and the optical images of the surgical site into the combined view further includes combining the second functional images with the first functional images and the optical images.

15. The method according to claim 13, further comprising extending a field of view of the camera with the second imager.

16. A method of visualizing a surgical site on a display, the method comprising:
positioning a camera within a surgical site to capture optical images along a first optical path;
positioning a first imager within the surgical site to capture first functional images along a second path separate from the first optical path; and
viewing a combined view of the first functional images overlaid the optical images on a display.

17. The method according to claim 16, further comprising positioning the first imager within the surgical site with a surgical instrument.

18. The method according to claim 17, wherein positioning the first imager includes positioning the first imager on an outer surface of an endoscope supporting the camera.

19. The method according to claim 16, further comprising actuating a switch to activate the combined view before viewing the combined view.
